(19) United States
(12) Patent Application Publication
(10) Pub. No.: US 2026/0108179 A1
(43) Pub. Date: Apr. 23, 2026

(54) APPARATUS AND METHOD FOR EXPECTING SPATIOTEMPORAL GAIT FACTOR

(71) Applicant: KOREA INSTITUTE OF SCIENCE AND TECHNOLOGY, Seoul (KR)

(72) Inventors: Kyung-Ryoul MUN, Seoul (KR); Jinwook KIM, Seoul (KR); Ankhzaya JAMSRANDORJ, Seoul (KR); Youn Jin CHUNG, Seoul (KR); Dawoon JUNG, Seoul (KR)

(73) Assignee: KOREA INSTITUTE OF SCIENCE AND TECHNOLOGY, Seoul (KR)

(21) Appl. No.: 19/363,798

(22) Filed: Oct. 21, 2025

(30) Foreign Application Priority Data
Oct. 22, 2024 (KR) 10-2024-0144970

(51) Int. Cl. A61B 5/11; G06V 10/62; G06V 10/77; G06V 40/20; G16H 50/20

(52) U.S. Cl. CPC A61B 5/112; G06V 10/62; G06V 10/7715; G06V 40/25; G16H 50/20

(57) ABSTRACT

The apparatus for expecting spatiotemporal gait factor according to an embodiment includes one or more processors; and a memory storing an instruction performed by the processors, in which the processors are configured to input one or more real-time image frames, in which a gait of a subject is captured, to a first model to extract image features of the real-time image frames, input the image features to a second model to extract temporal features of the real-time image frames, input the image features and the temporal features to a third model to extract spatial features of the real-time image frames, input the temporal features to a fourth model to output a probability distribution of gait events, predict a temporal factor of the gait based on the probability distribution of the gait events, and predict a spatial factor of the gait based on the spatial features.

### CROSS-REFERENCE TO RELATED APPLICATIONS

The present application claims priority to Korean Patent Application No. 10-2024-0144970, filed on Oct. 22, 2024, the entire contents of which is incorporated herein for all purposes by this reference.

### BACKGROUND OF THE INVENTION

### Field of the Invention

The disclosed embodiments relate to an apparatus and method for expecting spatiotemporal gait factor, and more particularly, to a technology for predicting spatiotemporal gait factor that is robust to capturing conditions of an input image.

### Explanation on State-Supported Research and Development

This study was conducted with the support of the Ministry of Science and ICT [Project Number: 1711192863, Subproject Number: RS-2022-00164554, Project Name: Development of Foot Diagnosis and Production Technology for Disabled Shoe].

This study was conducted with the support of the Ministry of Science and ICT [Project Number: 1711139131, Subproject Number: KMDF_PR_20200901_0101-04, Project Name: (Participation 3) Development of AI-based Personalized Exercise Management Platform and Service for Improving Muscle Function and Preventing Muscle Loss in Middle-Aged Group].

### Description of the Related Art

A gait factor is a health indicator used in various medical departments including rehabilitation medicine, orthopedics, internal medicine, and neurology. However, this gait factor requires a special sensor.

In this case, the special sensor should be handled only by experts through a complex and cumbersome experimental process. Therefore, there is a problem in that it is difficult for individuals to handle the special sensor in everyday life. In other words, the current technology for extracting gait factor has subjective limitations.

Objectively, the existing technology utilizing the special sensor is limited in a position and angle of a camera. Only input images captured from a camera at a specific angle and position may be used as analysis targets.

In other words, the gait factor is gaining prominence as an indicator that may monitor sports and personal health management beyond rehabilitation assistance, but accessibility to the gait factor is still insufficient.

### DOCUMENT OF RELATED ART

Korean Patent No. 10-2357770 (Published on Feb. 4, 2022)

### SUMMARY OF THE INVENTION

The disclosed embodiments provide a technology for predicting spatiotemporal gait factor.

According to an embodiment, an apparatus for expecting spatiotemporal gait factor includes: one or more processors; and a memory storing an instruction performed by the one or more processors, in which the one or more processors are configured to input one or more real-time image frames, in which a gait of a subject is captured, to a first model to extract image features of the real-time image frames, input the image features to a second model to extract temporal features of the real-time image frames, input the image features and the temporal features to a third model to extract spatial features of the real-time image frames, input the temporal features to a fourth model to output a probability distribution of gait events, predict a temporal factor of the gait based on the probability distribution of the gait events, and predict a spatial factor of the gait based on the spatial features.

The one or more processors may sequentially arrange the temporal factor and the spatial factor in step order to generate a sequence feature having a size of M×N. (in this case, M is a type of factor, and N is the number of factors arranged in time order.)

The one or more processors may input the sequence feature to a fifth model to evaluate a physical disease including at least one of a musculoskeletal disease and a neurological disease of the subject.

The one or more real-time image frames may be generated by being captured by a single camera that is freely disposed regardless of capturing conditions with the subject.

The one or more processors may output the probability distribution of the gait events, in which at least one of a heel and a forefoot of one foot or both feet of the subject touches or falls off the ground, using the fourth model.

At least one of the first model to the fourth model may be trained by training data labeled along with a time point of the gait event and coordinate information of the gait, as images captured by a plurality of cameras disposed at different locations and/or angles.

The one or more processors may extract at least one of a stride, a step, a stance phase, a swing phase, an early double-limb support phase, a terminal double-limb support phase, and a single-limb support phase as the temporal factor based on the probability distribution of the gait events.

The one or more processors may predict a spatial factor including at least one of a stride length, a step length, and a step width as the spatial factor based on the spatial feature.

According to another embodiment, a method for predicting spatiotemporal gait factor performed by an apparatus for expecting spatiotemporal gait factor including one or more processors and a memory storing an instruction performed by the one or more processors includes: inputting one or more real-time image frames, in which a gait of a subject is captured, to a first model to extract image features of the real-time image frames; inputting the image features to a second model to extract temporal features of the real-time image frames; inputting the image features and the temporal features to a third model to extract spatial features of the real-time image frames; inputting the temporal features to a fourth model to output a probability distribution of gait events; predicting a temporal factor of the gait based on the probability distribution of the gait events; and predicting a spatial factor of the gait based on the spatial features.

The method may further include sequentially arranging the temporal factor and the spatial factor in step order to generate a sequence feature having a size of M×N (in this case, M is a type of factor, and N is the number of factors arranged in time order.)

The method may further include inputting the sequence feature to a fifth model to evaluate a physical disease including at least one of a musculoskeletal disease and a neurological disease of the subject.

The one or more real-time image frames may be generated by being captured by a single camera that is freely disposed regardless of capturing conditions with the subject.

In the outputting of the probability distribution of the gait events, the probability distribution of the gait events, in which at least one of a heel and a forefoot of one foot or both feet of the subject touches or falls off the ground, using the fourth model may be output.

At least one of the first model to the fourth model may be trained by training data labeled along with a time point of the gait event and coordinate information of the gait, as images captured by a plurality of cameras disposed at different locations and/or angles.

The extracting of the temporal factor may include extracting at least one of a stride, a step, a stance phase, a swing phase, an early double-limb support phase, a terminal double-limb support phase, and a single-limb support phase as the temporal factor based on the probability distribution of the gait events.

The predicting of the spatial factor may include predicting a spatial factor including at least one of a stride length, a step length, and a step width as the spatial factor based on the spatial feature.

According to the disclosed embodiments, it is possible to automatically determine the spatiotemporal gait factors and physical diseases for real-time gait images freely under the capturing conditions without human intervention.

### BRIEF DESCRIPTION OF THE DRAWINGS

FIG. 1 is a block diagram for describing an apparatus for expecting spatiotemporal gait factor according to an embodiment.

FIG. 2 is a diagram for describing architecture of a learning model of an example.

FIG. 3 is a diagram for describing a probability distribution of gait events, which is an example.

FIG. 4 is a flowchart for describing a method of expecting spatiotemporal gait factor according to an embodiment.

### DETAILED DESCRIPTION OF INVENTION

The detailed descriptions are provided to help a comprehensive understanding of methods, apparatuses and/or systems described herein. However, embodiments are described by way of examples only and the present disclosure is not limited thereto.

In describing embodiments, when a detailed description of well-known technology related to the present disclosure may unnecessarily make unclear the gist of the embodiments invention, a detailed description thereof will be omitted.

The following terms are defined in consideration of the functions in the present disclosure and may be construed in different ways by the intention of users and operators. Therefore, the definitions thereof should be construed based on the contents throughout the specification. The terms used in the detailed description is merely for describing the embodiments and should in no way be limited.

Unless explicitly used otherwise, expressions in a singular form include the meaning in a plural form. It is natural that the terms first, second, etc., are only used to distinguish between various components and are not limited by the terms.

Meanwhile, an apparatus of the present disclosure may be entirely hardware, or may be partially hardware and may have aspects that are partially software. For example, a system for predicting severity of cognitive impairment of the elderly of this specification and each unit included therein may collectively refer to an apparatus for transmitting and receiving data of a specific format and contents in an electronic communication manner and software related thereto. In this specification, the terms such as “unit,” “module,” “server,” “system,” “apparatus,” or “terminal” are intended to refer to a combination of hardware and software driven by the hardware. For example, the hardware herein may be a data processing device including a CPU or other processor. In addition, software driven by hardware may refer to a running process, object, executable file, thread of execution, program, etc.

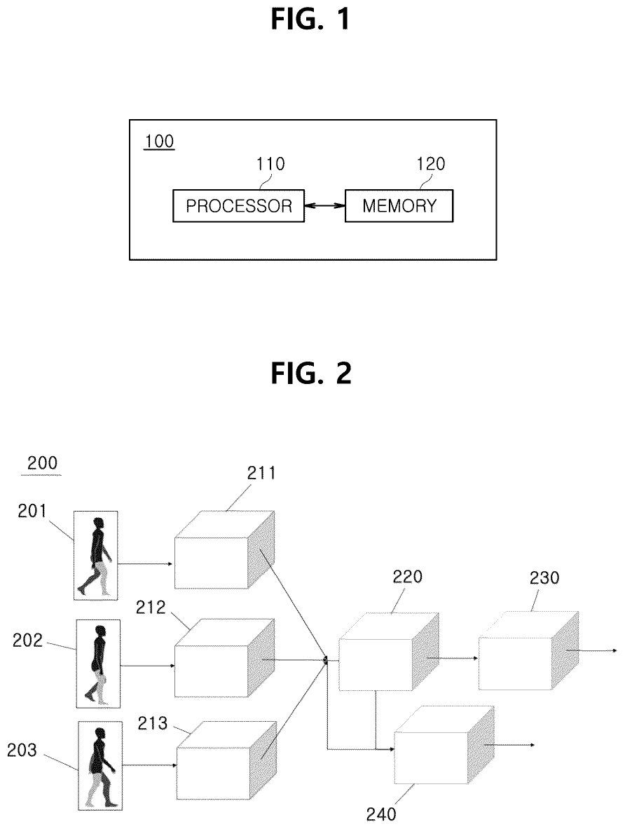

FIG. 1 is a block diagram for describing an apparatus 100 for expecting spatiotemporal gait factor according to an embodiment.

Referring to FIG. 1, the apparatus 100 for expecting spatiotemporal gait factor includes a processor 110 and a memory 120.

The processor 110 inputs one or more real-time image frames, in which a gait of a subject is captured, to a first model to extract image features of the real-time image frames.

Here, one or more real-time image frames may refer to images captured and generated by a single camera that is freely disposed regardless of capturing conditions with the subject.

When there are multiple real-time image frames, the processor 110 may sequentially input each real-time image frame to the corresponding first model to separately extract geometric features for each frame.

The processor 110 inputs image features to a second model to extract temporal features of the real-time image frames. The processor 110 may process temporal changes of one or more real-time image frames and extract the temporal features therefrom.

The processor 110 inputs the image features and temporal features to a third model to extract spatial features of the real-time image frames.

Specifically, the processor 110 may input a vector in which the image features and temporal features are combined (fused) to the third model to extract the spatial features of the real-time image frames.

The processor 110 inputs the temporal features to a fourth model to output a probability distribution of gait events.

The processor 110 may output the probability distribution of the gait events, in which at least one of a heel and a forefoot of one or both feet of a subject touches or falls off ground, using the fourth model.

For example, the processor 110 may output a probability distribution of a heel strike (HS) when at least one of left and right heels of a subject touches ground and a toe off (TO) when toes are lifted using a learning model.

The processor 110 predicts a temporal factor of gait based on the probability distribution of the gait events.

Here, the temporal factor may refer to a gait variable that changes over time. For example, the temporal factor may include variables related to a gait speed, a gait cycle, and a gait step length.

As a specific example, the temporal factor may include at least one of a stride, a step, a stance phase, a swing phase, an early double-limb support phase, a terminal double-limb support phase, and a single-limb support phase.

The processor 110 may predict the temporal factor from an event occurrence time point identified based on the probability distribution of the gait events.

The processor 110 predicts a spatial factor of gait based on the spatial features.

Here, the spatial factor may refer to a variable related to a spatial change that occurs during a gait motion. For example, the spatial feature may include at least one of a stride length, a step length, and a step width.

The processor 110 may evaluate physical diseases of a subject based on a sequence feature of the real-time image frame.

Here, the processor 110 may sequentially arrange the expected temporal factor and spatial factor in step order to generate sequence features having a size of M×N.

In this case, the processor 110 may generate the sequence features having the size of M×N by cross-arranging the expected temporal factor and spatial factor in the direction of both feet in step order.

The processor 110 may continuously generate the sequence features by moving windows having a certain size.

For example, the processor 110 may sequentially arrange an nth window and an n+1th window to continuously generate the sequence features.

For example, when an nth stride time R.Striden by the right foot, an n+1th stride time R.Striden+1 by the right foot, and an n+2th stride time R.Striden+2 by the right foot, and an nth stride time L.Striden by the left foot, an n+1th stride time L.Striden+1 by the left foot, and an n+2th stride time L.Striden+2 by the left foot are sequentially arranged in the nth window, an nth sequence feature, which is composed of a total of six stride times R.Striden, L.Striden, R.Striden+1, L.Striden+1, R.Striden+2, and L.Striden+2 and has a size of 1×6, may be acquired.

As another example, when the n+1th stride time R.Striden+1 by the right foot, the n+2th stride time R.Striden+2 by the right foot, and an n+3th stride time R.Striden+3 by the right foot, and the n+1th stride time R.Striden+1 by the left foot, the n+2th stride time R.Striden+2 by the right foot, and the n+3th stride time R.Striden+3 by the right foot are sequentially arranged in the n+1th window, the n+1th stride sequence feature, which is composed of a total of six stride times R.Striden+1, L.Striden+1, R.Striden+2, L.Striden+2, R.Striden+3, and L.Striden+3 and has a size of 1×6, may be acquired.

In this case, M may be a type of factor, and N may be the number of factors arranged in time order.

Specifically, the processor 110 may input the sequence features into a fifth model to evaluate at least one of a musculoskeletal disease and a neurological disease of a subject as a physical disease.

FIG. 2 is a diagram for describing architecture of a learning model of an example.

Referring to FIG. 2, the learning model includes first models 211, 212, and 213, a second model 220, a third model 230, and a fourth model 240.

The first models 211, 212, and 213 are models based on a convolutional neural network (CNN) and may be trained to extract geometric features of input images 201, 202, and 203. In this case, the first models 211, 212, and 213 may sequentially receive the plurality of input images 201, 202, and 203 as input sequences to extract unique geometric features of each input image.

The input images 201, 202, and 203 may be images generated from at least one of a plurality of motion cameras and general cameras disposed at various angles and locations.

The input images 201, 202, and 203 may be labeled along with correct values of the temporal factor and/or spatial factor.

For example, the input images 201, 202, and 203 may be labeled with a time point of a gait event identified from three-dimensional (3D) coordinate information of a marker attached to a body of a subject, as the correct value of the temporal factor.

As another example, the input images 201, 202, and 203 may be labeled along with the correct values of the spatial factors including a spatial stride distance, a step distance, a stride width, a gait speed, and a step speed identified from the 3D coordinate information of the marker attached to the body of the subject.

Meanwhile, the input images 201, 202, and 203 used as training data may include various images preprocessed by applying an augmentation technique to prevent overfitting.

In this way, the learning model may separately train unique geometric features of different input images 201, 202, and 203 through the plurality of first models 211, 212, and 213, and clearly recognize spatiotemporal changes of each image.

The second model 220 is a transformer-based model, and may be trained to extract temporal features by receiving geometric features of each input image. For example, the second model 220 can be trained to extract the temporal features by receiving a vector in which the geometric features of the input images are concatenated, respectively. The second model 220 may also be trained to extract the temporal features by receiving data in which relative location information of the plurality of input images, which are the input sequences, is encoded.

The third model 230 is a classification model including a fully-connected layer (FC layer), and may output a probability distribution of the gait events corresponding to the temporal features.

The fourth model 240 is a transformer-based model, and may be trained to extract the spatial features over time. The fourth model 240 may be trained to extract the spatial features by receiving input data generated by fusing the image features extracted from the first models 211, 212, and 213 and the temporal features extracted from the second model 220.

Meanwhile, here, the first models 211, 212, and 213 are trained using a set of three images as training data, which is an example. The number of training data input as the set may be different, and accordingly, the number of first models 211, 212, and 213 included in the learning model may also be provided corresponding to the number of training data input as the set.

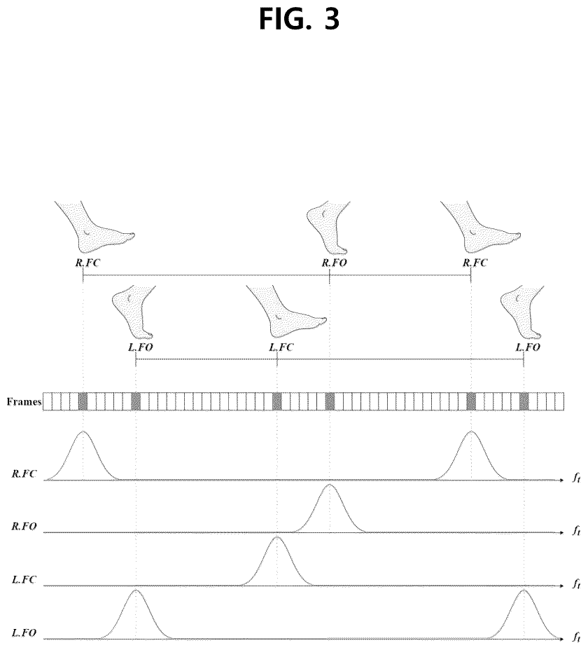

FIG. 3 is a diagram for describing an example of a probability distribution of gait events.

Referring to FIG. 3, the probability distribution of the gait events according to the gait of the subject output by the apparatus 100 for expecting spatiotemporal gait factor according to an embodiment is illustrated.

One or more real-time image frames used as the input data of the apparatus 100 for expecting spatiotemporal gait factor may be generated by continuously capturing the gait of the subject.

In this case, the apparatus 100 for expecting spatiotemporal gait factor may output the probability distribution of the gait events corresponding to the gait of the subject through the learning model.

Specifically, the apparatus 100 for expecting spatiotemporal gait factor may input a real-time image frame or sensor information corresponding to the real-time image frame to the learning model together to output the probability distribution of the gait events.

In this case, the apparatus 100 for expecting spatiotemporal gait factor may predict a probability distribution of a heel strike R.FC and toe off R.FO of a right foot, or a heel strike L.FC and toe off L.FO of a left foot as the gait event.

The apparatus 100 for expecting spatiotemporal gait factor according to an embodiment may not only identify the time point when the gait event occurs, but also predict the occurrence probability of the gait event for each frame.

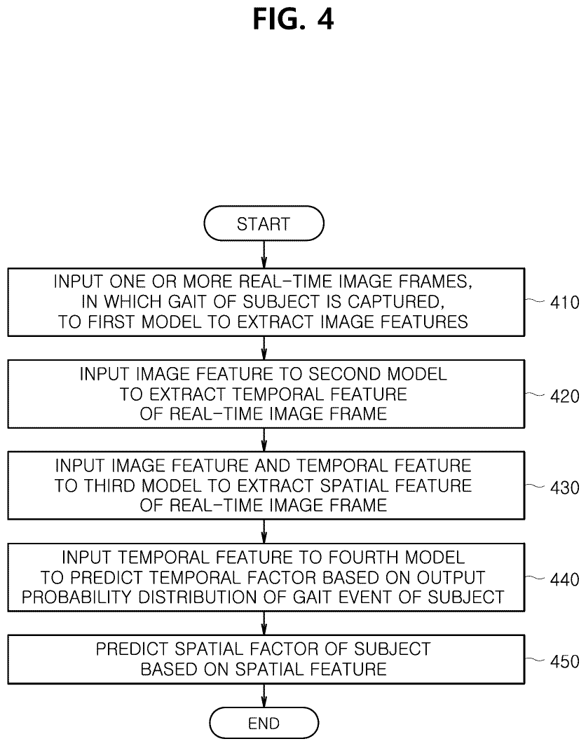

FIG. 4 is a flowchart for describing a method of expecting spatiotemporal gait factor according to an embodiment.

Referring to FIG. 4, the method of FIG. 4 may be performed by the apparatus 100 for expecting spatiotemporal gait factor of FIG. 1.

First, the apparatus 100 for expecting spatiotemporal gait factor inputs one or more real-time image frames in which the gait of the subject is captured to the first model to extract the image features of the real-time image frames (410).

The apparatus 100 for expecting spatiotemporal gait factor inputs the image features to the second model to extract the temporal features of the real-time image frames (420).

The apparatus 100 for expecting spatiotemporal gait factor inputs the image features and the temporal features to the third model to extract the spatial features of the real-time image frames (430).

The apparatus 100 for expecting spatiotemporal gait factor inputs the temporal features to the fourth model to output the probability distribution of gait events of a subject (440).

The apparatus 100 for expecting spatiotemporal gait factor predicts the temporal factor of the gait based on the probability distribution of the gait events (450).

The apparatus 100 for expecting spatiotemporal gait factor predicts the spatial factor of the gait based on the spatial features (460).

Meanwhile, in FIG. 4, the method is described by dividing into a plurality of steps, but at least some of the steps are performed in reverse order, combined with other steps, performed together, omitted, divided into detailed steps. Alternatively, one or more steps not illustrated may be added and performed.

### DESCRIPTION OF REFERENCE NUMERALS

100: APPARATUS FOR EXPECTING SPATIOTEMPORAL GAIT FACTOR
110: PROCESSOR
120: MEMORY
211, 212, 213: FIRST MODEL
220: SECOND MODEL
230: THIRD MODEL
240: FOURTH MODEL

What is claimed is:

1. An apparatus for expecting spatiotemporal gait factor, comprising:
one or more processors; and
a memory storing an instruction performed by the one or more processors,
wherein the one or more processors are configured to input one or more real-time image frames, in which a gait of a subject is captured, to a first model to extract image features of the real-time image frames,
input the image features to a second model to extract temporal features of the real-time image frames,
input the image features and the temporal features to a third model to extract spatial features of the real-time image frames,
input the temporal features to a fourth model to output a probability distribution of gait events,
predict a temporal factor of the gait based on the probability distribution of the gait events, and
predict a spatial factor of the gait based on the spatial features.

2. The apparatus of claim 1, wherein the one or more processors sequentially arrange the temporal factor and the spatial factor in step order to generate a sequence feature having a size of M×N.
(in this case, M is a type of factor, and N is the number of factors arranged in time order.)

3. The apparatus of claim 2, wherein the one or more processors input the sequence feature to a fifth model to evaluate a physical disease including at least one of a musculoskeletal disease and a neurological disease of the subject.

4. The apparatus of claim 1, wherein the one or more real-time image frames are generated by being captured by a single camera that is freely disposed regardless of capturing conditions with the subject.

5. The apparatus of claim 1, wherein the one or more processors outputs the probability distribution of the gait events, in which at least one of a heel and a forefoot of one foot or both feet of the subject touches or falls off the ground, using the fourth model.

6. The apparatus of claim 1, wherein at least one of the first model to the fourth model is trained by training data labeled along with a time point of the gait event and coordinate information of the gait, as images captured by a plurality of cameras disposed at different locations and/or angles.

7. The apparatus of claim 1, wherein the one or more processors extract at least one of a stride, a step, a stance phase, a swing phase, an early double-limb support phase, a terminal double-limb support phase, and a single-limb support phase as the temporal factor based on the probability distribution of the gait events.

8. The apparatus of claim 1, wherein the one or more processors predict a spatial factor including at least one of a stride length, a step length, and a step width as the spatial factor based on the spatial feature.

9. A method for expecting spatiotemporal gait factor performed by an apparatus for expecting spatiotemporal gait factor including one or more processors and a memory storing an instruction performed by the one or more processors, the method comprising:
inputting one or more real-time image frames, in which a gait of a subject is captured, to a first model to extract image features of the real-time image frames;
inputting the image features to a second model to extract temporal features of the real-time image frames;
inputting the image features and the temporal features to a third model to extract spatial features of the real-time image frames;
inputting the temporal features to a fourth model to output a probability distribution of gait events;
predicting a temporal factor of the gait based on the probability distribution of the gait events; and
predicting a spatial factor of the gait based on the spatial features.

10. The method of claim 9, further comprising sequentially arranging the temporal factor and the spatial factor in step order to generate a sequence feature having a size of M×N,
(in this case, M is a type of factor, and N is the number of factors arranged in time order).

11. The method of claim 10, further comprising inputting the sequence feature to a fifth model to evaluate a physical disease including at least one of a musculoskeletal disease and a neurological disease of the subject.

12. The method of claim 9, wherein the one or more real-time image frames are generated by being captured by a single camera that is freely disposed regardless of capturing conditions with the subject.

13. The method of claim 9, wherein in the outputting of the probability distribution of the gait events, the probability distribution of the gait events, in which at least one of a heel and a forefoot of one foot or both feet of the subject touches or falls off the ground, using the fourth model is output.

14. The method of claim 9, wherein at least one of the first model to the fourth model is trained by training data labeled along with a time point of the gait event and coordinate information of the gait, as images captured by a plurality of cameras disposed at different locations and/or angles.

15. The method of claim 9, wherein the extracting of the temporal factor includes extracting at least one of a stride, a step, a stance phase, a swing phase, an early double-limb support phase, a terminal double-limb support phase, and a single-limb support phase as the temporal factor based on the probability distribution of the gait events.

16. The method of claim 9, wherein the predicting of the spatial factor includes predicting a spatial factor including at least one of a stride length, a step length, and a step width as the spatial factor based on the spatial feature.
